(19) United States
(12) Patent Application Publication
(10) Pub. No.: US 2007/0298077 A1
(43) Pub. Date: Dec. 27, 2007

(54) CONSUMABLE VETERINARY MEDICINE DELIVERY DEVICE

(76) Inventor: Bradley E. Jones, Naples, NY (US)

Correspondence Address: JOHN M. HAMMOND;PATENT INNOVATIONS LLC, 150 LUCIUS GORDON DRIVE, SUITE 205, WEST HENRIETTA, NY, 14586, US

(21) Appl. No.: 11/425,664

(22) Filed: Jun. 21, 2006

(51) Int. Cl. A61K 47/00

(52) U.S. Cl. 424/439

(57) ABSTRACT

A medicine delivery device for oral administration of a medication, the device comprising an edible outer shell including a container piece comprised of a wall bounded by an outer surface, an inner surface, and a mating surface, and a recessed cavity formed within the inner surface; and a cap piece comprised of a cap wall partially bounded by a mating surface; and an edible paste comprising a first portion disposable on at least one of the mating surfaces of the container piece and the cap piece, and a second portion disposable within the recessed cavity of the container piece. A medicament is embedded in the second portion of edible paste, and the mating surfaces of the cap piece and the container piece are joined by the first portion of the edible paste, thereby forming a medicament cavity within the edible outer shell, within which the medicament is contained.

This invention relates in one embodiment to a device for delivery of orally dosed medication, and more particularly to a device that can be consumed by an animal that is being treated with the medication.

### BACKGROUND OF THE INVENTION

1. Field of the Invention

A consumable device for containing medication to be administered to an animal, the device comprising an edible outer shell enclosing a cavity within which the medication is disposed.

2. Description of Related Art

Many canines, felines, and other domesticated or captive animals are difficult to medicate orally. These animals can be very resistant to the new or unpleasant tastes and textures that are often intrinsic to oral medications. Since forced or manipulated feeding can be uncomfortable for both the caregiver and the animal, many caregivers resort to home remedies to hide or disguise the medicine. Common home remedies include gravy, bread, apples, peanut butter, and table scraps.

However, such solutions are frequently rejected by the animal and thus fail to deliver the complete prescribed dosage. The animal is often clever enough to separate the medication and spit it out, while enjoying the foodstuff in which it was embedded. In other instances, the animal may consume a part of the medication dose, and spit out the remainder. The caregiver is left with no quantitative idea of how much of the dose was administered, and thus how much more to administer to attain the correct dose. The caregiver must then consider the risks of overdosing and underdosing the medication in deciding how to proceed.

These problems can lead to serious health issues for the animal when antibiotics or other regimen drugs are being administered. These home remedy solutions have the additional disadvantage of adding incremental calories and inappropriate food products to the animal's diet.

Since it is not practical to modify the actual medication (due to the cost of custom preparations), there is a need for a medication delivery device that easily and consistently administers the medication to the animal, is an acceptable addition to the animal's diet, and reinforces a positive relationship between the caregiver and the animal.

Accordingly, It is an object of this invention to provide a consumable medication device for containing and delivering an oral medication to an animal.

It is a further object of this invention to provide a method for delivering an oral medication to an animal using a consumable medication device.

### SUMMARY OF THE INVENTION

In accordance with the present invention, there is provided a medicine delivery device for oral administration of a medication, the device comprising an edible outer shell including a container piece comprised of a container wall bounded by an outer surface, an inner surface, and a mating surface, and a recessed cavity formed within the inner surface; and a cap piece comprised of a cap wall partially bounded by a mating surface; and an edible paste comprising a first portion disposable on at least one of the mating surfaces of the container piece and the cap piece, and a second portion disposable within the recessed cavity of the container piece. Prior to administering the device (typically to an animal), a medicament is at least partially embedded in the second portion of edible paste, and the mating surface of the cap piece is joined to the mating surface of the container piece by the first portion of the edible paste, thereby forming a medicament cavity within the edible outer shell, within which the medicament is contained.

The container piece and the cap piece may consist essentially of an edible dough, and preferably a baked dough, which may have a malleable consistency. The paste preferably has a viscosity of a first value when at rest, which decreases when the paste is subjected to a shear condition, and which is restored when the shear condition is ceased, so that the paste is spreadable on the mating surface of one or both shell pieces, and so that the medicament can be embedded in the paste. The paste is preferably also highly viscoelastic and highly adherent to the mating surfaces of the pieces, so that it is effective in joining the pieces to each other. The paste may include ingredients selected from the group consisting of fish, beef, poultry, cheese, fruit, nuts, vegetables, vegetable oils, fats, emulsifiers, antioxidants, preservatives, water, broths, fruit juices, vegetable juices, sugars, colorants, flavors, gum arabic, vitamins, minerals, and pharmaceutical agents.

The container piece may have a variety of shapes, including hemispherical, semi-elliptical, and cylindrical. The cap piece may have a similar shape to the container piece, or the cap piece may have a cylindrical shape of low aspect ratio, i.e. a disk shape.

In accordance with the present invention, there is further provided a method for delivering an oral medication to an animal including a first step of providing components of a medicine delivery device including an edible outer shell comprised of a container piece having a container wall bounded by an outer surface, an inner surface, and a mating surface, and a recessed container cavity formed within the inner surface of the container wall; and a cap piece comprised of a cap wall partially bounded by a mating surface; and an edible paste. A first portion of the edible paste is disposed on at least one of the mating surfaces of the container piece and the cap piece, and a second portion of the edible paste is disposed within the recessed cavity of the container piece. A medicament is embedded at least partially within the second portion of the edible paste within the recessed cavity of the container piece. The mating surface of the container piece, the mating surface of the cap piece, and the first portion of the edible paste on at least one of the mating surfaces of the container piece and the cap piece are contacted with each other, thereby joining the container piece to the cap piece to provide a medicament-containing medicine delivery device. The delivery device including the medicament is administered orally to the animal.

### BRIEF DESCRIPTION OF THE DRAWINGS

The invention will be described by reference to the following drawings, in which like numerals refer to like elements, and in which:

FIG. 1 is a cross sectional illustration of an edible outer shell of the medicine delivery device of the present invention, comprising hemispherical shaped container and cap pieces;

FIG. 2 is a cross sectional illustration of the outer shell of the medicine delivery device shown in FIG. 1, wherein a first portion of edible paste has been disposed on the mating surface of the container piece;

FIG. 3 is a cross sectional illustration of the outer shell of the medicine delivery device shown in FIG. 2, wherein a second portion of edible paste has been disposed in the recessed cavity of the container piece, and a third portion of edible paste has been disposed in the recessed cavity of the cap piece;

FIG. 4 is a cross sectional illustration of the medicine delivery device shown in FIG. 3, wherein a medicament has been partially embedded in the second portion of edible paste in the cavity of the container piece;

FIG. 5 is a cross sectional illustration of the medicine delivery device shown in FIG. 4, wherein the mating surface of the container piece, the mating surface of the cap piece, and the first portion of the edible paste on the mating surface of the container piece have been contacted with each other, thereby joining the container piece to the cap piece to provide a medicament-containing medicine delivery device;

FIG. 6 is a cross-sectional illustration of an alternative medicine delivery device comprising an approximately hemispherical container piece including a cavity and a cylindrical disk-shaped cap piece;

FIG. 7 is a cross-sectional illustration of an alternative medicine delivery device comprising an ellipsoidal container piece including a cavity and a frusto-conical plug-shaped cap piece;

FIG. 8 is a cross-sectional illustration of the medicine delivery device of FIG. 5, further comprising a packaging film disposed between the container piece and the cap piece to provide a pre-packaged medicine delivery device; and

FIG. 9 is a cross-sectional illustration of a box containing a plurality of the pre-packaged medicine delivery devices of FIG. 8.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements. As used herein, the terms “edible” and “consumable” are used interchangeably, and are meant to indicate suitability for oral consumption by an animal.

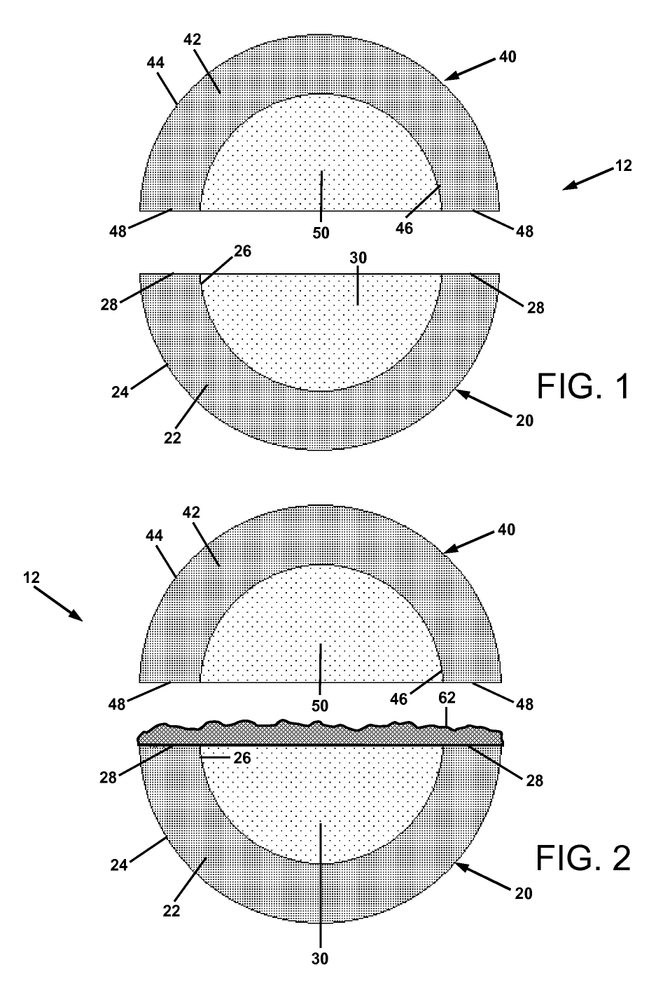

FIG. 1 is a cross sectional illustration of an edible outer shell of the medicine delivery device of the present invention, comprising hemispherical shaped container and cap pieces. Edible outer shell 12 is comprised of container piece 20 and cap piece 40. Container piece 20 is comprised of a wall 22 bounded by an outer surface 24, an inner surface 26, and a mating surface 28, and a recessed cavity 30 formed within the inner surface 26. Container piece 20 may have a variety of shapes, including but not limited to hemispherical, semi-elliptical, and cylindrical. In the embodiment depicted in FIG. 1, container piece 20 is approximately hemispherical.

Cap piece 40 is comprised of a wall 42 partially bounded by a mating surface 48. Cap piece 40 may have a similar shape to container piece 20, or the cap piece may have a cylindrical shape of low aspect ratio, i.e. a disk shape. In the embodiment depicted in FIG. 1, cap piece 40 is approximately hemispherical as container piece 20, and is comprised of wall 42 bounded by an outer surface 44, an inner surface 46, and a mating surface 48, and a recessed cavity 50 formed within the inner surface 42.

The container piece 20 and the cap piece 40 may consist essentially of an edible dough. The dough is preferably a baked dough, but may also be fried, or cooked via other known processes in the cooking arts. The edible dough is provided with a texture or consistency that may range from soft or malleable to stiff or crunchy. The specific consistency for a given application may be determined by the species or breed preference for a foodstuff, as well as the individual animal's regular diet.

As used herein, the term “malleable” is meant to indicate capable of being shaped or formed by pressure. A synonym in this context that is more commonly used in reference to a foodstuff is “chewy.” A bagel is an example of a food stuff with a chewy, or malleable consistency. A malleable consistency in the container piece and the cap piece is beneficial in that when the animal receives the medicine delivery device, the animal can deform the outer shell of the device with its teeth into a shape more conducive to swallowing. The outer shell may deform sufficiently to fracture partially, but not to an extent sufficient that the medicament contained in the device (to be described subsequently herein) is fractured and releases flavors or smells unpleasant to the animal.

Alternatively, the edible dough of the container piece 20 and cap piece 40 has a crumbly, or crisp and crunchy consistency. Examples of such doughs include pie crust, potato or corn chips, and cereal flakes. The use of a crumbly dough may be beneficial in instances in which the dough itself contains strong flavorants and odor releasing substances, which provide a sudden burst of flavor and smell that may mask the taste and smell of the medicament contained in the device.

The size and thickness of the container piece 20 and cap piece 40 of the outer shell 12 will vary depending upon the size and species of the animal receiving the medication, and upon the size of the medicament to be administered. Typically, the general size of the medicine delivery device will vary from about one half inch in diameter to about one and one half inches in diameter. The small size would be useful for medicating felines and small canines, and the large size would be useful for medicating e.g., large canines, swine, ovines, bovines, and equines.

The dough of container piece 20 and cap piece 40 may be prepared from a variety of ingredients depending on the specific intended end use. Common ingredients for the dough include but are not limited to flour (such as wheat, oat, corn, rice, and/or barley flour), bran, vegetable oil, animal fat, water, broth, egg, sweetener, and natural or artificial colors and/or flavorings.

The medicine delivery device further comprises an edible paste that acts as an adhesive to hold the container piece 20 and the cap piece 40 together when the device is fully assembled and ready to administer to an animal. FIG. 2 is a cross sectional illustration of the outer shell 12 of the medicine delivery device shown in FIG. 1, wherein a first portion of edible paste 62 has been disposed on the mating surface 28 of the container piece 20. Alternatively or additionally, the first portion of edible paste 62 may be disposed on the mating surface 48 of the container piece 40.

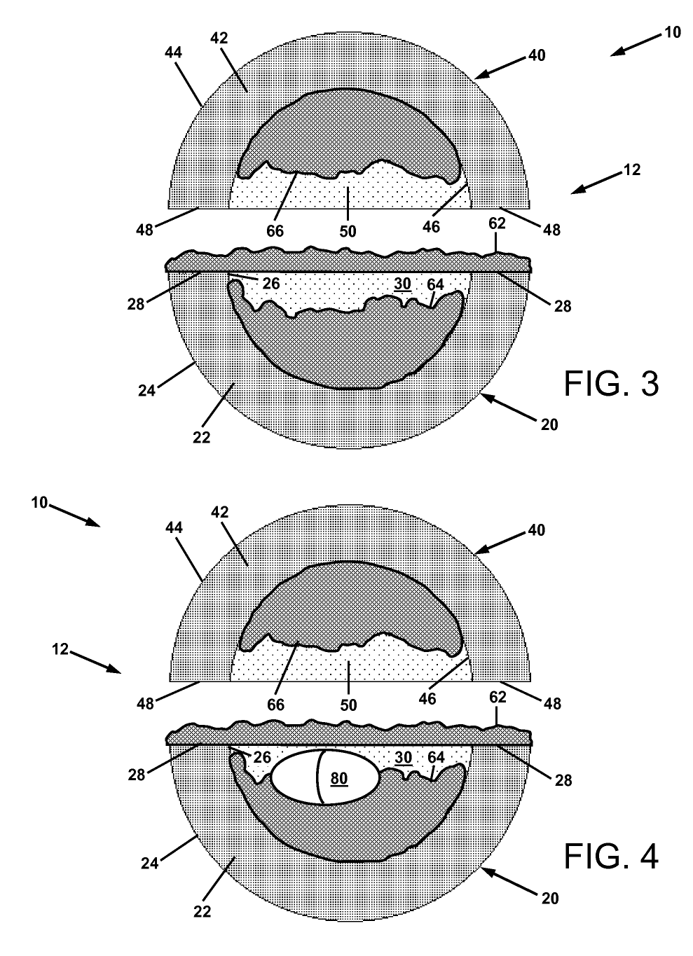

FIG. 3 is a cross sectional illustration of the outer shell of the medicine delivery device shown in FIG. 2, wherein a second portion of edible paste 64 has been disposed in the recessed cavity of the container piece 20; and FIG. 4 is a cross sectional illustration of the medicine delivery device 10 shown in FIG. 3, wherein a medicament 80 has been at least partially embedded in the second portion of edible paste 64 in the cavity 30 of the container piece 20. Medicament 80 may be pushed further into edible paste 64, and fully covered over with paste 64. Alternatively or additionally, a third portion of edible paste 66 may be disposed in the recessed cavity 50 of the cap piece 40, thereby further embedding the medicament 80 in the edible paste when the device 10 is fully assembled.

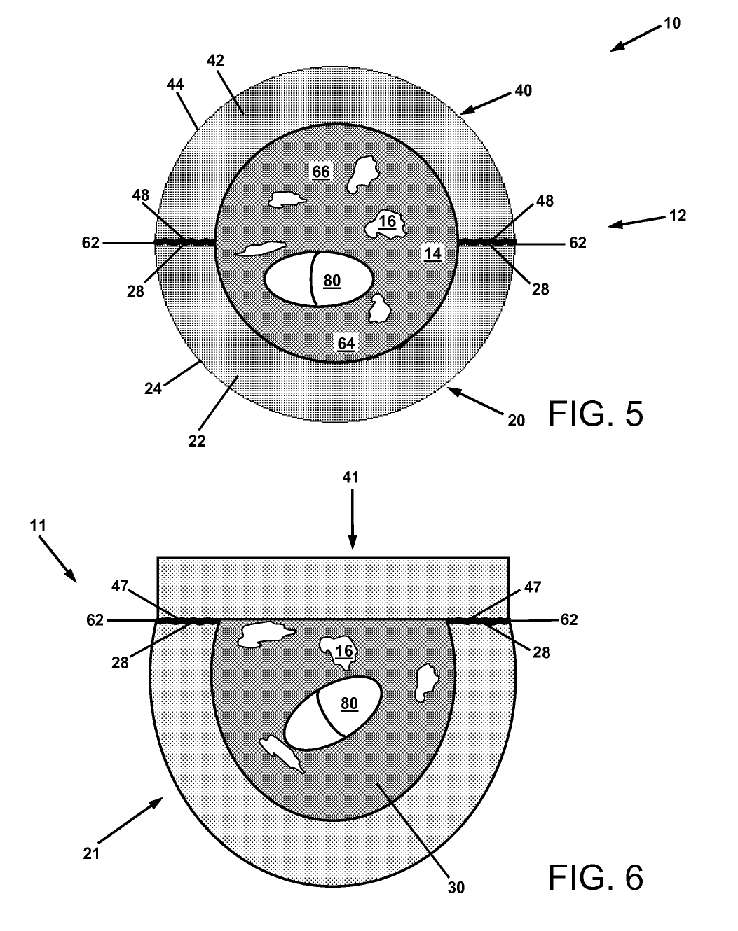

FIG. 5 is a cross sectional illustration of the medicine delivery device 10 shown in FIG. 4, but with device 10 in the fully assembled condition and ready to be administered to an animal. The mating surface 28 of the container piece 20, the mating surface 48 of the cap piece 40, and the first portion 62 of the edible paste have been contacted with each other, thereby joining the container piece 20 to the cap piece 40 of the medicine delivery device 10 containing medicament 80. Container cavity 30 and cap cavity 50 are contiguous with each other, thereby forming medicament cavity 14. It is noted that in the embodiment shown in FIG. 5, container piece 20 and cap piece 40 have been forced together such that some of the first portion 62 of edible paste has been extruded into the medicament cavity 14 formed within outer shell 12. Any excess extrudate forced outwardly (not shown) may be removed from the exterior of outer shell 12. It is also noted the thickness of the first portion 62 of edible paste is shown for illustrative purposes, and may be more or less than depicted. Additionally, more paste may be added to second portion 64 and/or third portion 66 to more fully fill medicament cavity 14 as shown in FIG. 5. Some small air pockets (e.g. air pocket 16) may also be entrained within the edible paste.

FIG. 6 is a cross-sectional illustration of an alternative medicine delivery device 11 comprising a semi-elliptical container piece 21 including a cavity 30 and a cylindrical disk-shaped cap piece 41. The mating surface 28 of the container piece 21, the mating surface 47 of the cap piece 41, and the first portion 62 of the edible paste have been contacted with each other, thereby joining the container piece 21 to the cap piece 41 of the medicine delivery device 11 containing medicament 80.

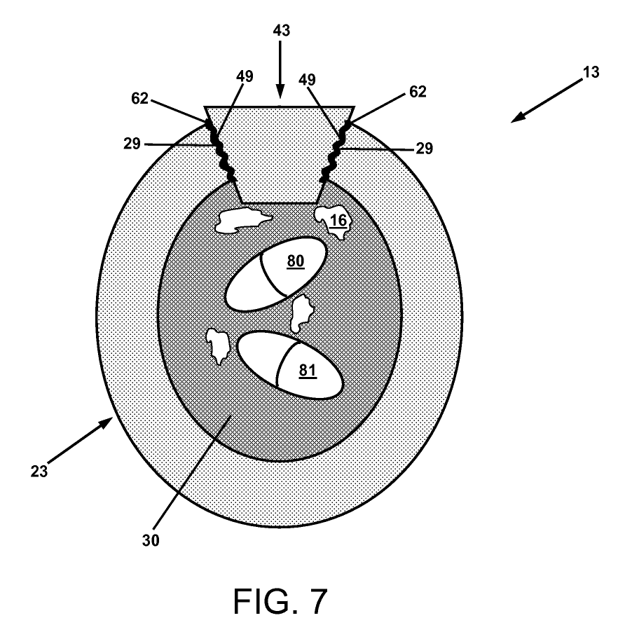

FIG. 7 is a cross-sectional illustration of an alternative medicine delivery device 13 comprising a semi-elliptical container piece 23 including a cavity 30 and a plug-shaped cap piece 43. Plug shaped piece 43 is preferably frusto-conical in shape, although other shapes may be used depending upon the shape of mating surface 29 of container piece 23. The mating surface 29 of the container piece 23, the mating surface 49 of the cap piece 43, and the first portion 62 of the edible paste have been contacted with each other, thereby joining the container piece 23 to the cap piece 43 of the medicine delivery device 13 containing medicament 80. Additionally, medicine delivery device contains a second medicament 81. It will be apparent that the medicament devices 10 and 11 shown in FIGS. 5 and 6 may also contain two or more medicaments.

It is to be understood that in the embodiments depicted in FIGS. 6 and 7, the semi-elliptical shape of container pieces 21 and 23 is meant to include a hemispherical shape.

In order to facilitate the dispensing and spreading of the paste onto the mating surface(s) of the container and or cap pieces, and into the recessed cavity of the container piece, certain Theological properties of the paste are preferred. The paste is preferably a semi-solid when at rest, and does not deform or flow when subjected to small forces, such as gravity. However, when subjected to sufficient shear and/or extensional forces such as by the act of extruding from a dispenser or spreading with a small tool, the paste behaves as a “shear thinning” and “extensional thinning” liquid, and deforms irreversibly. The paste preferably has a viscosity of a first value when at rest, which decreases when the paste is subjected to a shear and/or extensional condition, and which is restored when the shear and/or extensional condition is ceased. Accordingly, the paste is spreadable on the mating surface of one or both shell pieces 20 and 40, and the medicament 80 can be easily embedded in the paste. The paste is preferably also viscoelastic and adherent to the mating surfaces of the pieces, so that it is effective in joining the pieces 20 and 40 to each other. In terms more commonly used with reference to foodstuffs, the paste is “gummy” or “gooey.” Examples of edible substances with such properties are chewing gum and caramel.

The paste may be prepared from a variety of ingredients depending on the specific intended end use. The main ingredients comprise a ground preparation of meat, fish, poultry, cheese, fruit, nut, or vegetable (and combinations thereof). Additional ingredients may include oil, emulsifiers, anti-oxidants (e.g., butylated hydroxyanisole, butylated hydroxytoluene, tertiary butyl hydroquinone), natural preservatives, water, broth, juice, sweetener, and natural or artificial colors and flavorings, and gum arabic (acacia). Pharmaceuticals, vitamins, minerals, or other supplements) may also be included for specific applications. In another embodiment, the paste may contain one or more desired medications to be delivered, with the separate medicament 80 being optional. The paste may also be formulated with particularly strong smelling and/or tasting ingredients that will mask the smell or taste of the medicament contained therein, and that are attractive to the particular species of animal to be medicated. Examples of foodstuffs that have such ingredients are liverwurst meat, limburger cheese, and sardines.

Referring now to FIGS. 1-9, there is further provided a method for delivering an oral medication to an animal including a first step of providing components of a medicine delivery device 10 including an edible outer shell 12 comprised of a container piece 20 having a container wall 22 bounded by an outer surface 24, an inner surface 26, and a mating surface 28, and a recessed container cavity 30 formed within the inner surface 26 of the container wall 22; and a cap piece 40 comprised of a cap wall 42 partially bounded by a mating surface 48; and an edible paste. A first portion 62 of the edible paste is disposed on at least one of the mating surfaces 28 and 48 of the container piece 20 and the cap piece 40, respectively, and a second portion 64 of the edible paste is disposed within the recessed container cavity 30 of the container piece 20. A medicament 80 is embedded at least partially within the second portion 64 of the edible paste within the recessed container cavity 30 of the container piece 20. The mating surface 28 of the container piece 20, the mating surface 48 of the cap piece 40, and the first portion 62 of the edible paste on at least one of the mating surfaces 28/48 of the container piece 20 and the cap piece 40 are contacted with each other, thereby joining the container piece 20 to the cap piece 40 to provide a medicine delivery device 10 containing medicament 80. The delivery device 10 including the medicament 80 is administered orally to the animal. Alternatively, medicine delivery devices 11 and/or 13 of FIGS. 6 and 7 may be prepared in a similar manner with medicaments 80/81 and administered orally to the animal.

In one embodiment of the instant method for delivering an oral medication, the instant medicine delivery device 10 may be supplied in a packaged form such that the caregiver and administrator of the medication provides the selected medication and assembles the device as described herein and shown in FIGS. 1-5. In this embodiment, the instant method further includes the steps of removing the container piece 20, the cap piece 40, and the paste from the packaging (not shown). The paste may be provided in a small sealed cup (not shown) with a cover (not shown), which may be removed. The paste in the cup may be applied to the container piece and/or cap piece with a knife, spatula, or spoon. Alternatively, the paste may be provided in a tube (not shown) with a removable cap. The paste is dispensable by squeezing the tube.

The paste is applied to the container piece 20 and optionally to the cap piece 40 as described herein. The caregiver (not shown) inserts the desired medicament 80 into the paste-filled cavity 30 of the container piece 20 (or the cap piece 40 if paste has been dispensed into a cavity 50 therein), and presses the two pieces 20 and 40 back together. The caregiver then offers the device 10 containing the medication 80 to the animal (not shown), and the animal consumes the device 10 containing the medication 80. This embodiment provides safe and reliable delivery of prescribed or over-the-counter medications or supplements. It is convenient, easy to use, and cost effective.

In another embodiment of the instant method for delivering an oral medication, the instant medicine delivery device 10 is provided in a packaged form including medication contained therein as shown in FIG. 5. Device 10 is ready for administration to the animal. In this embodiment, the caregiver simply removes the device 10 from its packaging (not shown), and offers the device 10 containing the medication to the animal. The animal consumes the device 10 containing the medication. This embodiment provides safe and reliable delivery for various regimens of pharmaceuticals, vitamins, minerals, and supplements for dietary or medical purposes. The medication in device 10 may be comprised of a medicament 80, or the medication may comprise active ingredients that are pre-mixed in the edible paste at the time of manufacture. Exemplary applications for such a device and method may include administration of joint health, dental care, skin health, pain management, and specific dietary supplements.

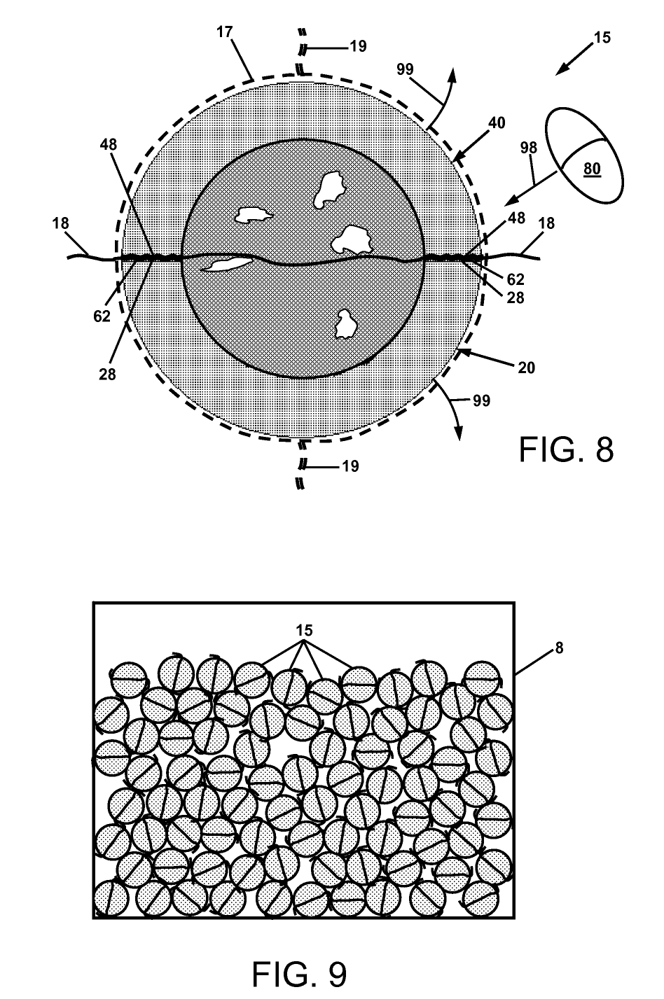

In another embodiment, the medicine delivery device is provided packaged in a nearly complete state, with only the medicament to be placed in the paste within the outer shell. This is best understood with reference to FIG. 8, which is a cross-sectional illustration of the medicine delivery device of FIG. 5, further comprising a packaging film disposed between the container piece and the cap piece to provide a pre-packaged medicine delivery device. In this embodiment, device 15 is provided to the caregiver fully assembled in individual packages. The individual packages may be comprised of a thin film wrapper 17 including a heat seal 19, shown in dotted line in FIG. 8. Container piece 20 and cap piece 40 are temporarily joined together at mating surfaces 28 and 48, but are separated by a peelable film 18. Peelable film may be made of any suitable material that is weakly adherent to the paste contained in device 15 such that peelable film 18 is removed without any paste, or with only a small amount of paste adhering thereto. Peelable film 18 may be made of a polymer such as polyethylene terephthalate. Peelable film 18 may be a metallized polymer film, of a metal foil such as aluminum foil.

To use device 10, the caregiver removes it from the packaging 19, and separates container piece 20 from cap piece 40 as indicated by arcuate arrows 99. The caregiver also completes the removal of peelable film 18 from either the cap piece 40 or the container piece 20 to which it remains partially adhered. Peelable film 18 is discarded. Medicament 80 is inserted into the paste in container piece 20 as indicated by arrow 98. Container piece 20 and cap piece 40 are rejoined together, and the fully assembled device 15 with medicament 80 is administered to the animal.

FIG. 9 is a cross-sectional illustration of a box 8 containing a plurality of the pre-packaged medicine delivery devices 15 of FIG. 8. It will be apparent that the devices 15 may be provided in a variety of other suitable packages, such as a bag, a can, a jar with a removable cap, and the like. It will be further apparent that delivery device may have the shape as shown for device 11 of FIG. 6, or device 13 of FIG. 7 as described previously herein.

It will be further apparent that the medicaments 80 and 81 depicted in FIGS. 4-9 are shown for illustrative purposes. Medicaments 80 and/or 81 may be provided in a variety of shapes, sizes, and configurations. Medicaments 80 and/or 81 may be formed as a pill, i.e. a pressed pellet of medication, with or without a coating. Medicaments 80 and/or 81 may comprise a multi-piece oblong hollow capsule, including first and second soluble pieces, within which a powdered or granular medicine is contained. Medicaments 80 and/or 81 may alternatively comprise more than one medication, and/or may comprise more than one pellet or capsule. Medicaments 80 and/or 81 may comprise a large number of small particles, even to the point wherein medicaments 80 and/or 81 are comprised of a fine powder dispersed in the edible paste. Thus, as used herein, a medicament “embedded” in the edible paste includes a range of medications from a single object to fine particles of medicine dispersed in the edible paste.

It will be further apparent that the use of the instant medicine delivery devices 10, 11, 13, and 15 are not limited to the cited felines, canines, swine, ovines, bovines, and equines. These species are to be considered as exemplary, and the instant device may be used to medicate many other species, including humans.

It is, therefore, apparent that there has been provided, in accordance with the present invention, a medicine delivery device for oral administration of a medication, and a method for delivering an oral medication to an animal. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

I claim:

1. A medicine delivery device for oral administration of a medication, the device comprising:
a. an edible outer shell including:
i. a container piece comprised of a container wall bounded by an outer surface, an inner surface, and a mating surface, and a recessed cavity formed within the inner surface; and
ii. a cap piece comprised of a cap wall partially bounded by a mating surface; and
b. an edible paste comprising a first portion disposable on at least one of the mating surfaces of the container piece and the cap piece, and a second portion disposable within the recessed cavity of the container piece.

2. The medicine delivery device as recited in claim 1, wherein the mating surface of the cap piece is joined to the mating surface of the container piece by the first portion of the edible paste, thereby forming a medicament cavity within the edible outer shell.

3. The medicine delivery device as recited in claim 2, further comprising a medicament at least partially embedded in the second portion of edible paste.

4. The medicine delivery device as recited in claim 2, wherein the container piece and the cap piece consist essentially of an edible dough.

5. The medicine delivery device as recited in claim 4, wherein the edible dough is malleable.

6. The medicine delivery device as recited in claim 2, further comprising a peelable film disposed between the cap piece and the container piece.

7. The medicine delivery device as recited in claim 1, wherein the paste has a viscosity of a first value when at rest, which decreases when the paste is subjected to a shear condition, and which is restored when the shear condition is ceased.

8. The medicine delivery device as recited in claim 1, wherein the paste is a viscoelastic paste.

9. The medicine delivery device as recited in claim 1, wherein the paste includes ingredients selected from the group consisting of fish, beef, poultry, cheese, fruit, nuts, vegetables, vegetable oils, fats, emulsifiers, antioxidants, preservatives, water, broths, fruit juices, vegetable juices, sugars, colorants, flavors, gum arabic, vitamins, minerals, and pharmaceutical agents.

10. The medicine delivery device as recited in claim 1, wherein the container piece and the cap piece are hemispherical in shape.

11. The medicine delivery device as recited in claim 1, wherein the container piece is semi-elliptical in shape, and the cap piece is a disk-shaped cylinder.

12. The medicine delivery device as recited in claim 1, wherein the container piece is substantially hemispherical in shape, and the cap piece is plug-shaped.

13. A medicine delivery device for oral administration of a medication, the device comprising:
a. an edible outer shell including:
i. a container piece comprised of a container wall bounded by an outer surface, an inner surface, and a mating surface, and a recessed container cavity formed within the inner surface of the container wall; and
ii. a cap piece comprised of a cap wall bounded by an outer surface, an inner surface, and a mating surface, and a recessed cap cavity formed within the inner surface of the cap wall;
b. an edible paste comprising a first portion disposed on at least one of the mating surfaces of the container piece and the cap piece, and a second portion disposed within the recessed cavity of the container piece.

14. The medicine delivery device as recited in claim 13, wherein the mating surface of the cap piece is joined to the mating surface of the container piece by the first portion of the edible paste and wherein the container cavity is contiguous with the cap cavity, thereby forming a medicament cavity within the edible outer shell.

15. The medicine delivery device as recited in claim 14, further comprising a medicament at least partially embedded in the second portion of edible paste.

16. The medicine delivery device as recited in claim 14, further comprising a peelable film disposed between the cap piece and the container piece.

17. The medicine delivery device as recited in claim 13, wherein the container piece and the cap piece consist essentially of an edible dough.

18. The medicine delivery device as recited in claim 17, wherein the edible dough is malleable.

19. The medicine delivery device as recited in claim 13, wherein the paste has a viscosity of a first value when at rest, which decreases when the paste is subjected to a shear condition, and which is restored when the shear condition is ceased.

20. The medicine delivery device as recited in claim 13, wherein the paste is a viscoelastic paste.

21. The medicine delivery device as recited in claim 13, wherein the paste includes ingredients selected from the group consisting of fish, beef, poultry, cheese, fruit, nuts, vegetables, vegetable oils, fats, emulsifiers, antioxidants, preservatives, water, broths, fruit juices, vegetable juices, sugars, colorants, flavors, gum arabic, vitamins, minerals, and pharmaceutical agents.

22. The medicine delivery device as recited in claim 13, wherein the container piece and the cap piece are semi-elliptical in shape.

23. The medicine delivery device as recited in claim 13, wherein the container piece and the cap piece are hemispherical in shape.

24. A method for delivering an oral medication to an animal comprising the steps of:
a. providing components of a medicine delivery device including:
i. an edible outer shell comprised of a container piece having a container wall bounded by an outer surface, an inner surface, and a mating surface, and a recessed container cavity formed within the inner surface of the container wall; and a cap piece comprised of a cap wall partially bounded by a mating surface; and
ii. an edible paste;
b. disposing a first portion of the edible paste on at least one of the mating surfaces of the container piece and the cap piece, and a second portion of the edible paste within the recessed cavity of the container piece;
c. embedding at least partially a medicament within the second portion of the edible paste within the recessed cavity of the container piece;
d. contacting the mating surface of the container piece, the mating surface of the cap piece, and the first portion of the edible paste on at least one of the mating surfaces of the container piece and the cap piece, thereby joining the container piece to the cap piece to provide a medicament-containing medicine delivery device; and
e. administering the medicament-containing medicine delivery device orally to the animal.

25. The method as recited in claim 24, wherein the medicine delivery device further comprises a peelable film disposed between the cap piece and the container piece.
